(19) United States
(12) Patent Application Publication
(10) Pub. No.: US 2025/0025054 A1
(43) Pub. Date: Jan. 23, 2025

(54) SYSTEMS AND METHODS FOR DETERMINING HEMODYNAMICS

(71) Applicant: Shanghai United Imaging Intelligence Co., Ltd., Shanghai (CN)

(72) Inventors: Yikang Liu, Cambridge, MA (US); Dehong Fang, Malden, MA (US); Lin Zhao, Billerica, MA (US); Zhang Chen, Brookline, MA (US); Xiao Chen, Lexington, MA (US); Shanhui Sun, Lexington, MA (US); Terrence Chen, Lexington, MA (US)

(73) Assignee: Shanghai United Imaging Intelligence Co., Ltd., Shanghai (CN)

(21) Appl. No.: 18/223,414

(22) Filed: Jul. 18, 2023

(51) Int. Cl. A61B 5/02; G16H 30/20

(52) U.S. Cl. CPC A61B 5/02007; G16H 30/20

(57) ABSTRACT

Described herein are systems, methods, and instrumentalities associated with automatic determination of hemodynamic characteristics. An apparatus as described may implement a first artificial neural network (ANN) and a second ANN. The first ANN may model a mapping from a set of 3D points associated with one or more blood vessels to a set of hemodynamic characteristics of the one or more blood vessels, while the second ANN may generate, based on a geometric relationship of the set of points in a 3D space, parameters for controlling the mapping. The apparatus may obtain a 3D anatomical model representing at least one blood vessel of a patient based on one or more medical images of the patient, and determine, based on the first ANN and the second ANN, a hemodynamic characteristic of the at least one blood vessel of the patient at a target location of the 3D anatomical model.

### BACKGROUND

Artery diseases such as aneurysms and coronary diseases caused by plaque buildup along the inner walls of coronary arteries are among the leading causes of deaths worldwide. Computational fluid dynamics may be used to diagnose and monitor these diseases by computing blood flow characteristics such as fractional flow reserves, wall shear stresses, etc. based on laws of physics and mathematical equations (e.g., using finite element and/or finite difference methods). These physics and mathematics based techniques, however, involve high computational costs and complexities, and are slow for clinical purposes.

### SUMMARY

Described herein are machine learning (ML) based on systems, methods, and instrumentalities associated with automatic determination of hemodynamic (e.g., blood flow) characteristics. An apparatus according to embodiments of the present disclosure may include a processor and a memory configured to store computer program instructions that, when executed by the processor, cause the processor to perform one or more of the following tasks. The processor may obtain, based on one or more medical images of a patient, a 3D anatomical model that represents at least one blood vessel of the patient, and determine, based on a first artificial neural network (ANN) and a second ANN, a hemodynamic characteristic of the at least one blood vessel of the patient at a target location of the 3D anatomical model. The first ANN may be configured to model a mapping from a set of points in the 3D anatomical model to a set of hemodynamic characteristics of the at least one blood vessel, while the second ANN may be configured to generate, based on a geometric relationship of the set of points in the 3D anatomical model, parameters for controlling, at least partially, the determination of the hemodynamic characteristic of the at least one blood vessel.

In examples, the mapping modeled by the first ANN may associate 3D coordinates and boundary conditions of each of the set of points in the 3D anatomical model with corresponding hemodynamic characteristics, the 3D anatomical model may indicate the 3D coordinates and boundary conditions a point of the 3D anatomical model at the target location, and the hemodynamic characteristic of the at least one blood vessel at the target location may be determined further based on the 3D coordinates and the boundary conditions of the point at the target location. In examples, the hemodynamic characteristic of the patient at the target location may include at least one of a pressure, a blood flow velocity, a blood flow rate, or a wall shear stress of the at least one blood vessel at the target location.

In examples, the first ANN may include a multilayer perceptron (MLP), the second ANN includes a graph neural network (GNN), and the GNN may be trained to learn the geometric relationship of the set of points in the 3D anatomical model based on a centerline of the at least one blood vessel. In examples, the MLP may be configured to determine one or more feature vectors associated with the target location, the parameters generated by the GNN may include respective weights to be applied to the one or more feature vectors, and the hemodynamic characteristic of the at least one blood vessel at the target location may be determined by applying the respective weights to the one or more feature vectors determined by the MLP.

In examples, the computer program instructions may, when executed by the processor, further cause the processor to obtain one or more physiological measurements (e.g., blood pressure, blood flow velocity, etc.) of the patient and adjust the mapping modeled by the first ANN based on the one or more physiological measurements. In examples, the training of at least one of the first ANN or the second ANN may include comparing a hemodynamic characteristic predicted by the ANN(s) to a hemodynamic characteristic determined based on a law of physics, and adjusting the parameters (e.g., weights) of at least one of the first ANN or the second ANN based on the comparison.

### BRIEF DESCRIPTION OF THE DRAWINGS

A more detailed understanding of the examples disclosed herein may be had from the following description, given by way of example in conjunction with the accompanying drawing.

FIG. 1 is a diagram illustrating an example of predicting the hemodynamic characteristics of one or more blood vessels of a patient using ML-based techniques.

FIG. 2 is a diagram illustrating an example of an ANN that may be used to predict the hemodynamics characteristics of one or more blood vessels of a patient based on a 3D anatomical model of the blood vessels.

FIG. 3 illustrates an example of a graphical neural network (GNN) that may be used to perform one or more of the tasks described herein.

FIG. 4 is a flow diagram illustrating example operations that may be associated with training a neural network to perform one or more of the tasks described herein.

FIG. 5 is a flow diagram illustrating example operations that may be associated with predicting the hemodynamic characteristics of one or more blood vessels at a target location and/or time.

FIG. 6 is a block diagram illustrating example components of an apparatus that may be configured to perform the tasks described herein.

### DETAILED DESCRIPTION

The present disclosure is illustrated by way of example, and not by way of limitation, in the figures of the accompanying drawings. A detailed description of illustrative embodiments will be provided with reference to the figures. Although these embodiments may be described with certain technical details, it should be noted that the details are not intended to limit the scope of the disclosure. Further, while some embodiments may be provided in the context of predicting specific hemodynamic properties, those skilled in the art will understand that the techniques disclosed in those embodiments may be applicable to the determination of all types of hemodynamic properties.

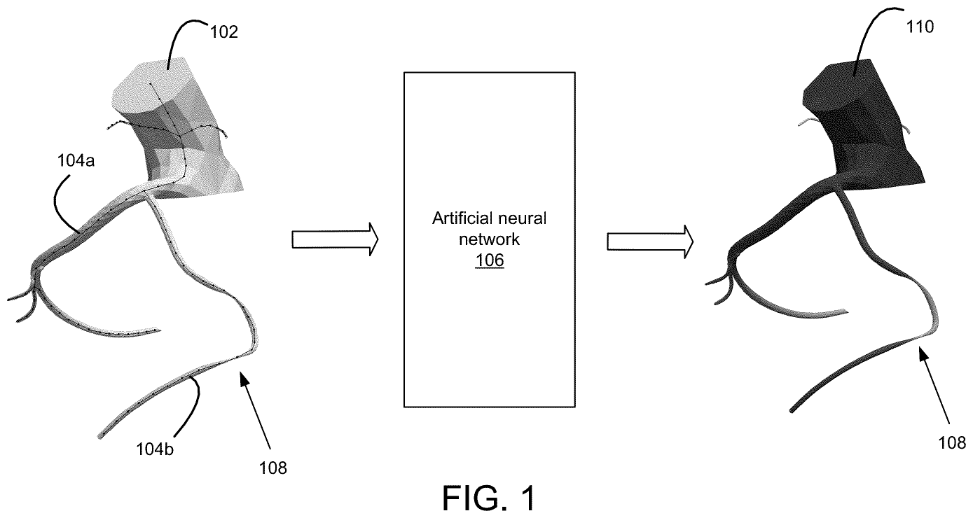

FIG. 1 illustrates an example of predicting the hemodynamic (e.g., blood flow) characteristics of one or more blood vessels of a patient using machine learning (ML) based techniques. As shown, the prediction of the hemodynamic characteristics may be made based on an anatomical model 102 of the one or more blood vessels (e.g., 104a and 104b of FIG. 1) and one or more artificial neural networks (ANNs) 106. The anatomical model 102 may be a three-dimensional (3D) model that may be derived based on one or more medical images of the patient depicting the blook vessels. The one or more medical images may include, for example, X-ray fluoroscopy images, computed tomography (CT) images, magnetic resonance imaging (MRI) images, ultra-sound images, etc., and the derivation of the anatomical model 102 may be accomplished using various computer-assisted visualization techniques including, for example, digital subtraction angiography (DSA) and/or vessel synthesis. For instance, to build the anatomical model 102, blood vessels may be imaged from multiple (e.g., two or more) views, and respective vessel masks and/or centerlines for the multi-view vessel images may be obtained using a suitable image processing technique. Subsequently, corresponding points on the respective centerlines of the multi-view vessel images may be annotated (e.g., manually or automatically), and an initial 3D anatomical model may be reconstructed based on camera parameters (e.g., from an X-ray scanner such as a C-arm machine) used to capture the multi-view vessel images, point correspondence, and/or vessel masks. The initial 3D model and camera parameters may then be optimized so that the 3D model's projection in each view may be consistent with the vessel mask associated with that view. Synthesized vessel data may be used to train a computer model for constructing the anatomical model 102. An example of a synthesis process may include generating (e.g., randomly) centerlines of a vessel tree and vessel radius at each point on the centerlines, and calculating vessel volumes and/or surface meshes based on the vessel centerlines and radius. Certain rules may be enforced to improve the resemblance of the synthesized data to real vessels. For example, at the distal ends of blood vessels, the angle of a vessel bifurcation may be limited within a specific range. The respective curvatures of one or more points on a vessel centerline may also be limited within a specific range. In addition, a vessel radius may be made smaller at sites that are closer to distal ends, and may be forced to be smooth along a vessel (e.g., except for occasional sudden decreases at sites of simulated stenosis).

The anatomical model 102 may include positional and/or geometric information about the blood vessels 104a and/or 104b in a 3D space. For example, the positional information may include (x, y, z) coordinates of a plurality of points in the 3D space that may be associated with the blood vessels, and the geometric information may indicate the respective lengths, sizes, and/or orientations of the blood vessels as well as their spatial relationships in the 3D space. For example, the geometric information captured in the anatomical model 102 may indicate the geometry of a vessel branch bearing a stenosis, such as, e.g., vessel radius, tortuosity measurements, calcification measurements, etc. of the vessel branch. As another example, the geometric information captured in the anatomical model 102 may indicate the geometry of a coronary artery tree, such as, e.g., left or right dominance, the size of coronary territories associated with myocardial masses, terminal radius of each coronary branch, bifurcations, trifurcations, etc. As yet another example, the geometric information captured in the anatomical model 102 may indicate one or more boundary conditions of the blood vessels, such as, e.g., blood inflow velocity at a point on an inlet cross section, outlet parameter models that may define the relationship between blood flow rates and blood pressures, etc.

The ANN 106 may be trained to model a mapping from a set of points in the anatomical model 102 (and/or a time value) to a set of hemodynamic characteristics of the blood vessels 104a and/or 104b represented by the anatomical model 102. The set of points may be associated with respective positional and/or geometric properties indicated by the anatomical model 102, the time value may be associated with a cardiac cycle (e.g., end of diastole, end of systole, etc.), and the hemodynamic characteristics of each point in the anatomical model may include at least one of a pressure, blood flow velocity, blood flow rate, or blood vessel wall shear stress that correspond to the positional and/or geometric properties of the point indicated by the anatomical model 102. As such, when given time, positional and/or geometric properties of a target location 108 (e.g., a location of interest) associated with a blood vessel (e.g., represented by anatomical model 102), ANN 106 may be used to determine (e.g., predict) the hemodynamic characteristics of the blood vessel at the target location based on the aforementioned mapping learned by the ANN 106 (e.g., via training). Such hemodynamic characteristics may include, for example, a fraction flow reserve (FFR) that may indicate the ratio of maximum achievable blood flow through a blockage (area of stenosis) of a blood vessel to the maximum achievable blood flow in the same vessel in the hypothetical absence of the blockage. The hemodynamic characteristics may also include, for example, a wall shear stress of a blood vessel that may indicate the force per unit area exerted by the wall of the vessel on the blood flow in a direction on a local tangent plane of the vessel. Once determined, these hemodynamic characteristics may be visualized, for example, via a computer-generated heatmap 110 that may be overlaid on the anatomical model 102, and used to make diagnostic decisions or suggestions for the patient.

ANN 106 may be trained to learn the mapping described herein using a training dataset that may include positional and/or geometric information about a blood vessel paired with hemodynamic characteristics of the blood vessel. The positional and/or geometric information may be obtained from blood vessel anatomical models and/or boundary conditions associated with the anatomical models. For example, using the DSA techniques described herein, anatomical models of blood vessels (e.g., geometric information) may be obtained. Using information encompassed in these anatomical models (e.g., blood flow velocities) and physiological measurements of specific individuals as well as the general population (e.g., blood pressures, ratios between blood pressures and flow rates at outlet, etc.), boundary conditions associated with the blood vessels may be determined. The anatomical models and the boundary conditions may then be used to train the ANN 106, with loss functions that may be designed based on computational fluid dynamics (CFD) laws. The training may involve, for example, extracting features associated with the blood vessels (e.g., via feature vectors) based on the anatomical models of the blood vessels, predicting hemodynamics characteristics of the blood vessels (e.g., pressures and/or velocities) at a target location based on the extracted features, and optimizing the parameters of the ANN 106, so that a predicted hemodynamics characteristic of the blood vessels may satisfy the relevant laws of physics (e.g., Navier-Stokes equations and/or other linear or non-linear partial differential equations). For example, a predicted velocity and/or pressure at a point may need to satisfy the Navier-Stokes equation, the ratio between a blood pressure and a flow rate at an outlet may be equal to a certain value (e.g., as a boundary condition at the outlet), the velocity at a vessel wall may be zero (e.g., substantially near zero), the velocity at an inlet may satisfy a given spatial distribution, etc.

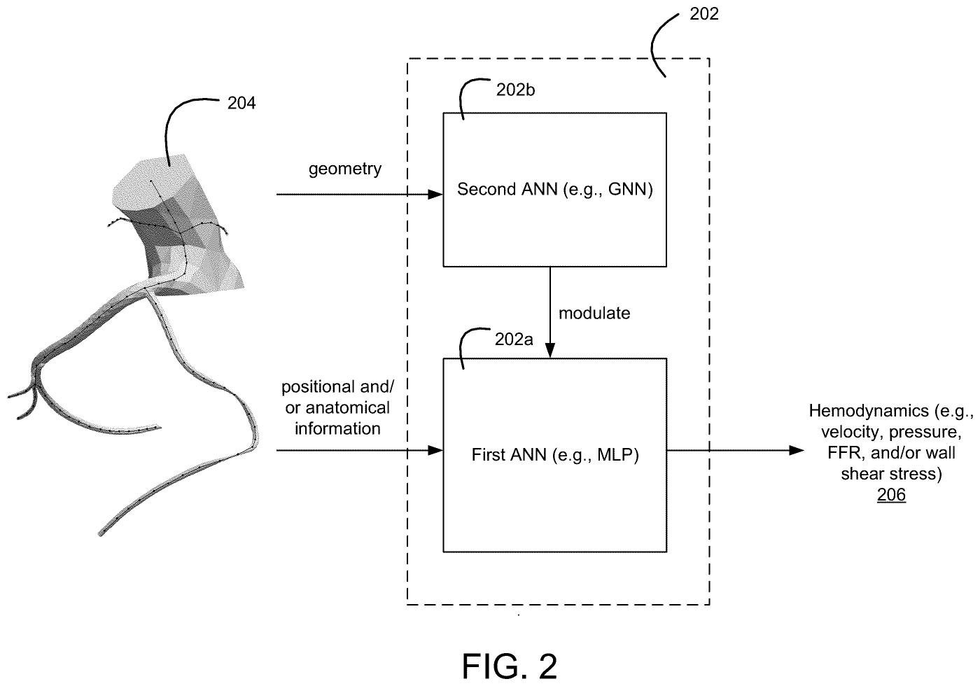

FIG. 2 illustrates an example of an artificial neural network 202 (e.g., ANN 106 of FIG. 1) that may be used to predict hemodynamics characteristics of one or more blood vessels (e.g., coronary arteries) of a patient based on a 3D anatomical model 204 of the blood vessels. As describe herein, the 3D anatomical model 204 may be derived based on one or more medical images of the patient, and may include positional (e.g., (x, y, z) coordinates of a plurality of points on the 3D model) and/or geometric (e.g., length, radius, orientation, etc.) information about the blood vessels. ANN 202 may include a first ANN 202a and a second ANN 202b. The first ANN 202a may be configured to model a mapping from a set of points of the anatomical model 204 to a set of hemodynamic characteristics of the blood vessels, while the second ANN 202b may be configured to generate a plurality of control parameters that may be used to control or modulate (e.g., at least partially) the derivation of the mapping from the set of points in the anatomical model 204 to the set of hemodynamic characteristics of the one or more blood vessels. For example, the first ANN 202a may include a multilayer perceptron (MLP) configured to extract features (e.g., positional and/or anatomical features) from the 3D anatomical model 204 and predict one or more hemodynamic characteristics 206 (e.g., blood flow velocity, pressure, FFR, and/or wall shear pressure) of the blood vessels at a given target location based at least on the extracted features. The second ANN 202b may include a graphical neural network (GNN) configured to determine the geometric relationships of multiple points on the anatomical model 204 and output a set of weights that may be applied to the features extracted by the MLP during the determination of hemodynamic characteristics 206 (and/or the learning of the mapping modeled by first ANN 202a). As will be described in greater detail below, the GNN may be trained to learn the geometry of the blood vessels along respective centerlines of the blood vessels and use an attention mechanism to determine (e.g., based on the geometric information encoded by the GNN) weights that may emphasize certain dimensions of the features extracted by the MLP while de-emphasize other dimensions of the features. This process may be represented, mathematically, by the following equations:

$${w}_{\vec{x}} = {g}_{θ} ( M )$$

$$y = {f}_{φ}^{( 2 )} ( {w}_{\vec{x}} \cdot {f}_{φ}^{( 1 )} ( \vec{x} , t ) )$$

where g_{θ} may represent the GNN with trainable parameters θ, M may represent the 3D anatomical model 204, and w_{\vec{x},t} may present the weights output by the GNN, whose values may depend on the corresponding location x on the 3D model M and/or a time t (e.g., a time spot within a cardiac cycle). Further, f_{φ} may represent the MLP with trainable parameters φ, f_{φ}⁽¹⁾(\vec{x},t) may represent a feature vector generated by a first part of the MLP, f_{φ}⁽²⁾ may represent a feature vector generated by a second part of the MLP, · may represent a channel wise multiplication operation, and y may represent intermediate features generated by the MLP or a hemodynamic characteristic predicted by the MLP. The equations above may represent the operations of two MLP layers, but the MLP may include more than two layers, with each layer modulated by w_{\vec{x}}·f_{φ}⁽¹⁾(\vec{x},t).

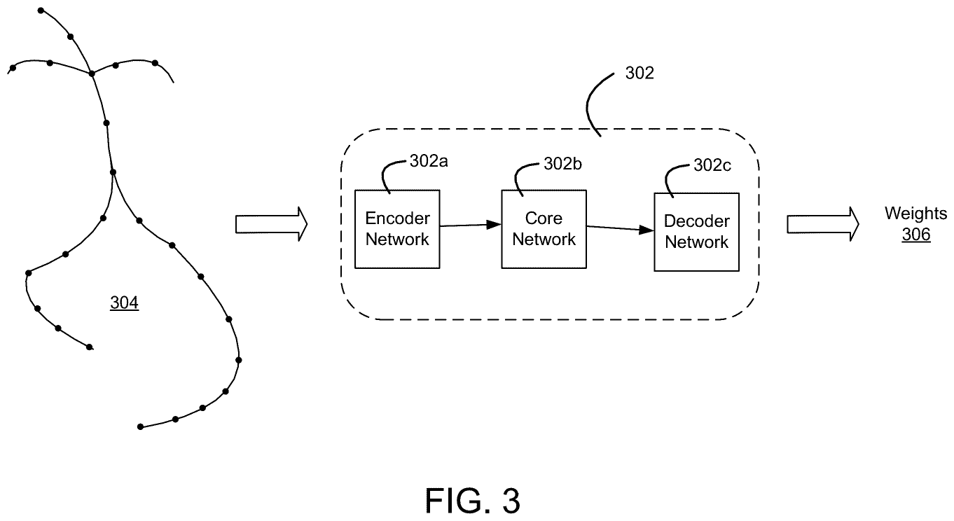

FIG. 3 illustrates an example of a graphical neural network (GNN) 302 (e.g., GNN 202b of FIG. 2) that may be used to generate weights to be applied to features extracted by an MLP (e.g., MLP 202a of FIG. 2) while predicting hemodynamic characteristics of one or more blood vessels. As shown in FIG. 3, GNN 302 may be used to encode the geometric relationships of the blood vessels (e.g., based on points 304 located along respective centerlines of the blood vessels) and determine weights 306 based on the encoded geometric relationships (e.g., by decoding the encoded information). GNN 302 may be suitable for performing such a task because, as illustrated by FIG. 3, points 304 may form a graph, where each node of the graph may correspond to a respective point and each edge of the graph may represent the geometric relationship of the nodes connected by the edge. As such, GNN 302 may, in examples, include an encoder network 302a, a core neural network 302b, and a decoder neural network 302c. The encoder neural network 302a may be trained to extract respective features associated with the nodes (e.g., points 304) and edges (e.g., spatial relationships of the points 304) of the graph representation, the core neural network 302b may be trained to estimate respective states of the nodes and edges of the graph representation based on the features extracted by the encoder neural network 302a, and the decoder neural network 302c may be trained to predict the weights 306 based on the respective states of the nodes and edges estimated by the core neural network 302b.

Each of the encoder neural network 302a, core neural network 302b, or decoder neural network 302c may include multiple convolutional layers, one or more pooling layers, and/or one or more fully-connected layers. Each convolutional layer may include a plurality of convolution kernels or filters with respective weights, the values of which may be learned through a training process so as to extract features from an input of the convolutional layer. The convolutional layer may be followed by batch normalization and/or linear or non-linear activation (e.g., such as rectified linear unit or ReLU activation), and the features extracted by the convolutional layer may be down-sampled through one or more pooling layers to obtain a representation of the features, for example, in the form of a feature map or feature vector. One or more of the encoder, core, and decoder neural networks may further include one or more un-pooling layers and one or more transposed convolutional layers. Through the un-pooling layers, the neural network may up-sample the features extracted from the input and further process the up-sampled features through the one or more transposed convolutional layers (e.g., via a plurality of deconvolution operations) to derive an up-scaled or dense feature representation. The dense feature representation may then be used to predict (e.g., hypothesize) the weights 306 to be applied by the MLP described herein.

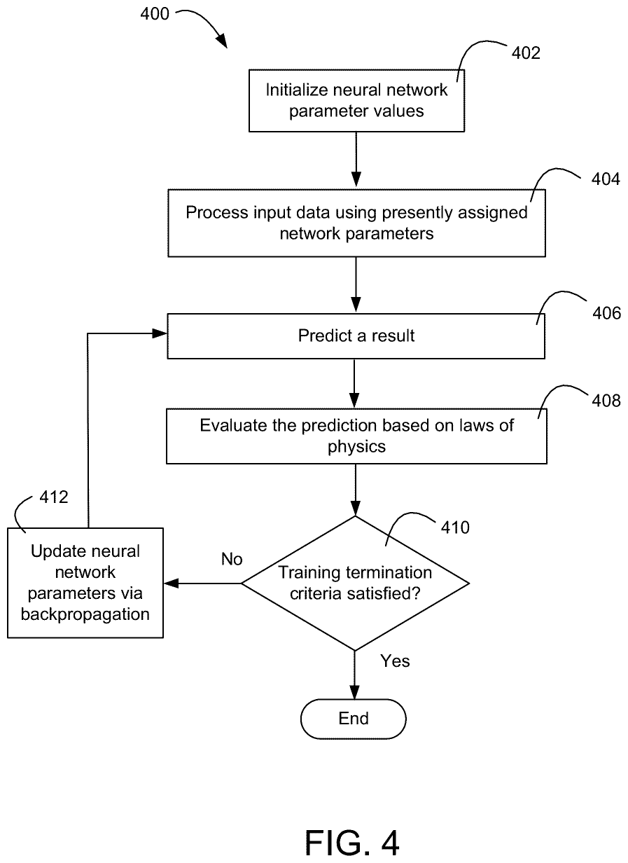

FIG. 4 illustrates example operations 400 that may be associated with training a neural network (e.g., the MLP 202a and/or GNN 202b of FIG. 2) to perform one or more of the tasks described herein. As shown, the training operations 400 may include initializing the operating parameters of the neural network (e.g., weights associated with various layers of the neural network) at 402, for example, by sampling from a probability distribution or by copying the parameters of another neural network having a similar structure. The training operations 400 may further include processing an input (e.g., positional, geometric and/or anatomical information about a blood vessel) using presently assigned parameters of the neural network at 404, and making a prediction for a desired result (e.g., a hemodynamic characteristic or weight used to calculate the hemodynamic characteristic) at 406. The prediction result may be compared to a result calculated based on one or more laws of physics (e.g., Navier-Stokes equations) to evaluate how well the neural network has performed based on its current parameters. At 410, a loss may be calculated based on the aforementioned comparison to determine whether one or more training termination criteria are satisfied. For example, the training termination criteria may be determined to be satisfied if the loss is below a threshold value or if the change in the loss between two training iterations falls below a threshold value. If the determination at 410 is that the termination criteria are satisfied, the training may end; otherwise, the presently assigned network parameters may be adjusted at 412, for example, by backpropagating a gradient descent of the loss function through the network, before the training returns to 406.

In examples, the parameters of the neural network learned via the training operations 400 (e.g., via an offline training process) and/or the result predicted by the neural network may be further refined based on patient-specific measurements (e.g., physiological measurements) and/or patient-specific geometries obtained during an online prediction operation (e.g., after the neural network has been deployed to perform a prediction task). Such patient-specific measurements and/or geometries may include, for example, a blood pressure (BP) of the patient measured using a BP monitor device and/or a blood flow velocity of the patient measured via X-ray fluoroscopy. Once obtained, these measurements and/or geometries may be used to adjust the prediction made by the neural network. The amount of adjustment (e.g., equivalent to a loss) may also be used to fine-tune the parameters of the neural network (e.g., via backpropagation) before the neural network is used for another prediction task.

For simplicity of explanation, the training operations 400 are depicted and described with a specific order. It should be appreciated, however, that the training operations 400 may occur in various orders, concurrently, and/or with other operations not presented or described herein. Furthermore, it should be noted that not all operations that may be included in the training method are depicted and described herein, and not all illustrated operations are required to be performed.

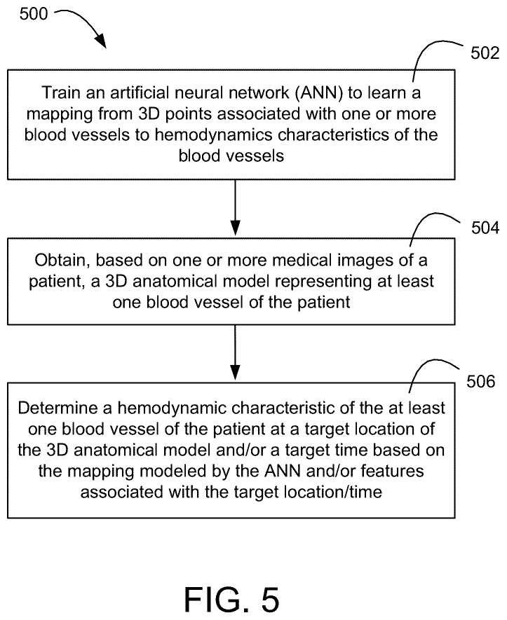

FIG. 5 illustrates example operations 500 that may be associated with predicting the hemodynamic characteristics of one or more blood vessels at a target location and/or time. As shown in FIG. 5, operations 500 may include training an artificial neural network (ANN) at 502 to learn a mapping from a set of points in a three-dimensional (3D) space associated with the one or more blood vessels to a set of hemodynamic characteristics of the one or more blood vessels. As described herein, the ANN may include a first ANN (e.g., an MLP) configured to extract features associated with the one or more blood vessels from a 3D anatomical model of the blood vessels and predict the hemodynamic characteristics of the blood vessels at the target location and/or time based on features related to the target location and/or time. The ANN may also include a second ANN (e.g., a GNN) configured to generate, based on a geometric relationship of the set of points in the 3D space, parameters that may be used to control, at least partially, derivation of the aforementioned mapping. Also as shown in FIG. 5, operations 500 may further include obtaining, based on one or more medical images of a patient, a 3D anatomical model representing at least one blood vessel of the patient at 504, and determining, at 506, a hemodynamic characteristic (e.g., blood flow velocity, pressure, etc.) of the at least one blood vessel of the patient at a target location of the 3D anatomical model and/or at a target time. The determination may be made, for example, based on the mapping modeled by the ANN as described herein and/or features associated with the target location and/or target time. From the determined hemodynamic characteristics, other blood vessel parameters and/or properties (e.g., such as FFR, wall shear stress, etc.) may be calculated.

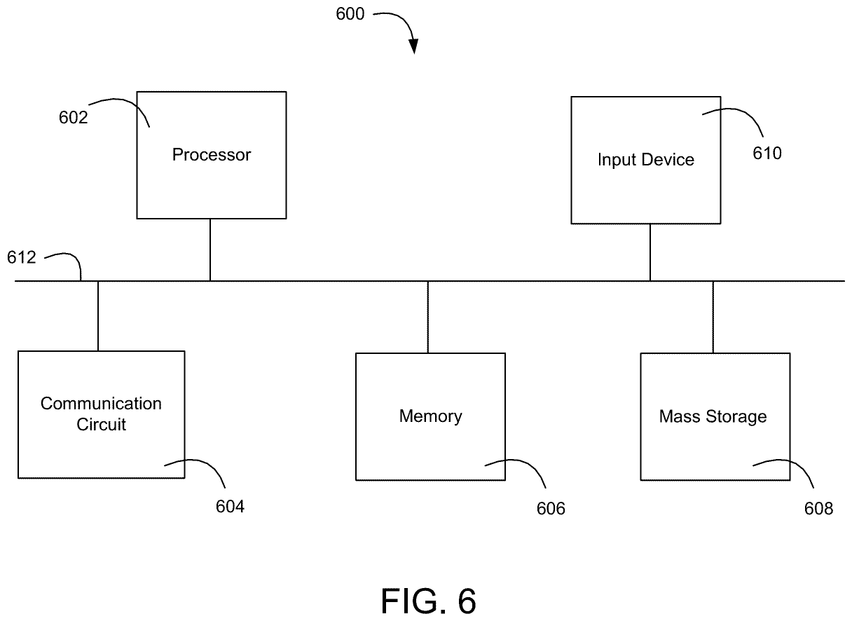

The systems, methods, and/or instrumentalities described herein may be implemented using one or more processors, one or more storage devices, and/or other suitable accessory devices such as display devices, communication devices, input/output devices, etc. FIG. 6 illustrates an example apparatus 600 that may be configured to perform the automatic image annotation tasks described herein. As shown, apparatus 600 may include a processor (e.g., one or more processors) 802, which may be a central processing unit (CPU), a graphics processing unit (GPU), a microcontroller, a reduced instruction set computer (RISC) processor, application specific integrated circuits (ASICs), an application-specific instruction-set processor (ASIP), a physics processing unit (PPU), a digital signal processor (DSP), a field programmable gate array (FPGA), or any other circuit or processor capable of executing the functions described herein. Apparatus 600 may further include a communication circuit 604, a memory 606, a mass storage device 608, an input device 610, and/or a communication link 612 (e.g., a communication bus) over which the one or more components shown in the figure may exchange information.

Communication circuit 604 may be configured to transmit and receive information utilizing one or more communication protocols (e.g., TCP/IP) and one or more communication networks including a local area network (LAN), a wide area network (WAN), the Internet, a wireless data network (e.g., a Wi-Fi, 3G, 4G/LTE, or 5G network). Memory 606 may include a storage medium (e.g., a non-transitory storage medium) configured to store machine-readable instructions that, when executed, cause processor 602 to perform one or more of the functions described herein. Examples of the machine-readable medium may include volatile or non-volatile memory including but not limited to semiconductor memory (e.g., electrically programmable read-only memory (EPROM), electrically erasable programmable read-only memory (EEPROM)), flash memory, and/or the like. Mass storage device 608 may include one or more magnetic disks such as one or more internal hard disks, one or more removable disks, one or more magneto-optical disks, one or more CD-ROM or DVD-ROM disks, etc., on which instructions and/or data may be stored to facilitate the operation of processor 602. Input device 610 may include a keyboard, a mouse, a voice-controlled input device, a touch sensitive input device (e.g., a touch screen), and/or the like for receiving user inputs to apparatus 600.

It should be noted that apparatus 600 may operate as a standalone device or may be connected (e.g., networked, or clustered) with other computation devices to perform the functions described herein. And even though only one instance of each component is shown in FIG. 6, a skilled person in the art will understand that apparatus 600 may include multiple instances of one or more of the components shown in the figure.

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure. In addition, unless specifically stated otherwise, discussions utilizing terms such as “analyzing,” “determining,” “enabling,” “identifying,” “modifying” or the like, refer to the actions and processes of a computer system, or similar electronic computing device, that manipulates and transforms data represented as physical (e.g., electronic) quantities within the computer system's registers and memories into other data represented as physical quantities within the computer system memories or other such information storage, transmission or display devices.

It is to be understood that the above description is intended to be illustrative, and not restrictive. Many other implementations will be apparent to those of skill in the art upon reading and understanding the above description. The scope of the disclosure should, therefore, be determined with reference to the appended claims, along with the full scope of equivalents to which such claims are entitled.

What is claimed is:

1. An apparatus, comprising:
a processor; and
a memory configured to store computer program instructions that, when executed by the processor, cause the processor to:
obtain, based on one or more medical images of a patient, a 3D anatomical model that represents at least one blood vessel of the patient; and
determine, based on a first artificial neural network (ANN) and a second ANN, a hemodynamic characteristic of the at least one blood vessel of the patient at a target location of the 3D anatomical model, wherein:
the first ANN is configured to model a mapping from a set of points in the 3D anatomical model to a set of hemodynamic characteristics of the at least one blood vessel, and the second ANN is configured to generate, based on a geometric relationship of the set of points in the 3D anatomical model, parameters for controlling, at least partially, the determination of the hemodynamic characteristic of the at least one blood vessel of the patient.

2. The apparatus of claim 1, wherein the hemodynamic characteristic of the at least one blood vessel of the patient at the target location includes at least one of a pressure, a blood flow velocity, a blood flow rate, or a wall shear stress of the at least one blood vessel at the target location.

3. The apparatus of claim 1, wherein the mapping modeled by the first ANN associates 3D coordinates and boundary conditions of each of the set of points in the 3D anatomical model with corresponding hemodynamic characteristics.

4. The apparatus of claim 3, wherein the 3D anatomical model indicates the 3D coordinates and boundary conditions of a point of the 3D anatomical model at the target location, and wherein the hemodynamic characteristic of the at least one blood vessel at the target location is determined further based on the 3D coordinates and the boundary conditions of the point at the target location.

5. The apparatus of claim 1, wherein the first ANN includes a multilayer perceptron (MLP) and the second ANN includes a graph neural network (GNN).

6. The apparatus of claim 5, wherein the GNN is trained to learn the geometric relationship of the set of points in the 3D anatomical model based on a centerline of the at least one blood vessel.

7. The apparatus of claim 5, wherein the MLP is configured to determine one or more feature vectors associated with the target location, wherein the parameters generated by the GNN include respective weights to be applied to the one or more feature vectors, and wherein the hemodynamic characteristic of the at least one blood vessel at the target location is determined by applying the respective weights generated by the GNN to the one or more feature vectors determined by the MLP.

8. The apparatus of claim 1, wherein, when executed by the processor, the computer program instructions further cause the processor to obtain one or more physiological measurements of the patient and adjust the mapping modeled by the first ANN based on the one or more physiological measurements.

9. The apparatus of claim 8, wherein the one or more physiological measurements of the patient include at least one of a blood pressure of the patient or a blood velocity of the patient.

10. The apparatus of claim 1, wherein, during training of at least one of the first ANN or the second ANN, a hemodynamic characteristic predicted based on the mapping modeled by the first ANN is compared to a hemodynamic characteristic determined based on a law of physics, and parameters of at least one of the first ANN or the second ANN are adjusted based on the comparison.

11. A method, comprising:
obtaining, based on one or more medical images of a patient, a three-dimensional (3D) anatomical model that represents at least one blood vessel of the patient; and
determining, based on a first artificial neural network (ANN) and a second ANN, a hemodynamic characteristic of the at least one blood vessel of the patient at a target location of the 3D anatomical model, wherein:
the first ANN is configured to model a mapping from a set of points of the 3D anatomical model to a set of hemodynamic characteristics of the at least one blood vessel, and the second ANN is configured to generate, based on a geometric relationship of the set of points in the 3D anatomical model, parameters for controlling, at least partially, the determination of the hemodynamic characteristic of the at least one blood vessel of the patient.

12. The method of claim 11, wherein the hemodynamic characteristic of the at least one blood vessel of the patient at the target location includes at least one of a pressure, a blood flow velocity, a blood flow rate, or a wall shear stress of the at least one blood vessel at the target location.

13. The method of claim 11, wherein the mapping modeled by the first ANN associates 3D coordinates and boundary conditions of each of the set of points in the 3D anatomical model with corresponding hemodynamic characteristics.

14. The method of claim 13, wherein the 3D anatomical model indicates the 3D coordinates and boundary conditions of a point of the 3D anatomical model at the target location, and wherein the hemodynamic characteristic of the at least one blood vessel at the target location is determined further based on the 3D coordinates and the boundary conditions of the point at the target location.

15. The method of claim 11, wherein the first ANN includes a multilayer perceptron (MLP) and the second ANN includes a graph neural network (GNN).

16. The method of claim 15, wherein the GNN is trained to learn the geometric relationship of the set of points in the 3D anatomical model based on a centerline of the at least one blood vessel.

17. The method of claim 15, wherein the MLP is configured to determine one or more feature vectors associated with the target location, wherein the parameters generated by the GNN include respective weights to be applied to the one or more feature vectors, and wherein the hemodynamic characteristic of the at least one blood vessel at the target location is determined by applying the respective weights generated by the GNN to the one or more feature vectors determined by the MLP.

18. The method of claim 11, further comprising obtaining one or more physiological measurements of the patient and adjusting the mapping modeled by the first ANN based on the one or more physiological measurements.

19. The method of claim 18, wherein the one or more physiological measurements of the patient include at least one of a blood pressure of the patient or a blood velocity of the patient.

20. A non-transitory computer-readable medium comprising instructions that, when executed by a processor included in a computing device, cause the processor to implement the method of claim 11.
